(19) United States
(12) Patent Application Publication
(10) Pub. No.: US 2005/0260769 A1
(43) Pub. Date: Nov. 24, 2005

(54) Handheld medical logging apparatus and method

(75) Inventors: Fredrik Jonsson, Mellbystrand (SE); Lars Olof Hagstrom, Helsingborg (SE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: HEMOCUE AB, Angelholm (SE)

(21) Appl. No.: 10/858,205

(22) Filed: Jun. 2, 2004

(30) Foreign Application Priority Data
May 24, 2004 (SE) 0401337-1

(51) Int. Cl. G01N 33/543

(52) U.S. Cl. 436/518

(57) ABSTRACT

A handheld medical logging apparatus for recording and logging data related to a person's blood glucose level comprises measuring means adapted to measure a glucose level, one or more buttons, and control circuitry coupled to the buttons. The control circuitry is adapted to, in response to an activation of a specific button, directly enter an event logging mode, in which mode an occurrence of events may be recorded in the apparatus, wherein each event may affect the blood glucose level and is represented by a predetermined code. The control circuitry is further, in the event logging mode, adapted to interpret activations of the buttons as the code representing an event and create a signal indicative of the occurred event. The apparatus further comprises a memory coupled to the control circuitry and adapted to receive the signal, and in response thereof record the occurrence of the event.

### TECHNICAL FIELD

The present invention relates to a handheld medical logging apparatus and a method for recording and logging data related to a person's blood glucose level, wherein the apparatus comprises measuring means adapted to measure a glucose level in a blood sample.

### BACKGROUND OF THE INVENTION

A person having diabetes has a shortage of insulin resulting in reduced regulation of glucose in blood. If the blood glucose level becomes too high or too low, it could have severe consequences for the diabetic. The diabetic will therefore need continuous supply of insulin in order to treat the disease.

The diabetic normally performs the administration of insulin himself. Thus, his daily life is minimally affected. The diabetic will naturally need instructions on how to perform the self-treatment. These instructions are based on advice from a physician. However, in order to give appropriate advice, the physician will need to know how the glucose level of the diabetic normally varies. Therefore, the diabetic will need to regularly perform tests and create a log of his glucose level.

U.S. Pat. No. 5,019,974 discloses a master computer, in which a treatment programme is created, and a recorder, which may be connected to the master computer for receiving the programme. The recorder prompts the patient to perform the actions according to the programme. The patient will then record the performed actions, including testing the blood glucose, in the recorder for review by the physician to adjust the programme. The patient must submit to the instructions of the programme requiring e.g. meals on specific times. The programme has no flexibility and does not allow the patient to vary from the strict programme of leading his lifestyle.

U.S. Pat. No. 4,731,726 provides a glucose monitor, which in connection with the patient making a blood glucose test prompts the patient to enter information related to the blood glucose level. Thus, the patient is requested to indicate events, such as exercise, emotional stress, illness and dietary intake, occurring since the last test. This implies that the patient needs to respond to several questions each time a blood glucose test is taken. This is time-consuming and could cause irritation, since the patient will need to respond to these questions several times a day. Moreover, the further information is merely stored in connection with the blood measurement and does therefore not indicate the specific time of occurrence of the events.

### SUMMARY OF THE INVENTION

It is an object of the invention to provide a diabetic with a simple way of recording glucose values and events affecting the values.

It is a further object to provide possibility to record relevant data such that an appropriate treatment programme may be created based on the lifestyle of the patient.

These objects of the invention are achieved by a handheld medical logging apparatus according to claim 1 and a method according to claim 8.

Thus, a handheld medical logging apparatus for recording and logging data related to a person's blood glucose level is provided. The apparatus comprises measuring means adapted to measure a glucose level in a blood sample. The apparatus further comprises one or more buttons, control circuitry operatively coupled to the one or more buttons and adapted to, in response to an activation of a specific button amongst the one or more buttons, directly enter an event logging mode, in which mode an occurrence of one or more events from a set of events may be recorded in the apparatus. Each of said events may affect the blood glucose level and is represented by a predetermined code. The control circuitry is, in the event logging mode, adapted to interpret one or more activations of the one or more buttons as the code representing an event and create a signal indicative of the occurred event. The apparatus further comprises a memory operatively coupled to the control circuitry and adapted to receive the signal and, in response thereof, record the occurrence of the event represented by the code.

Further, there is provided a method for recording and logging data related to a person's blood glucose level in a handheld medical logging apparatus comprising one or more buttons and a measuring means adapted to measure a glucose level in a blood sample. The method comprises receiving an indication in the apparatus that a specific button among the one or more buttons has been activated, and in direct response to the received indication, the apparatus entering an event logging mode, wherein an occurrence of one or more events from a set of events may be recorded in the apparatus, each of said events may affect the blood glucose level and is represented by a predetermined code. The method further comprises, in the event logging mode, receiving in the apparatus one or more button activations specifying one of the codes representing an event, wherein the occurrence of said event is recorded in the apparatus.

The invention provides a possibility to record data in a simple way. A diabetic, who needs to record events affecting the blood glucose level several times a day, will greatly benefit from a simple way of recording events. The recording of events is very simple, since the user, which is normally the diabetic, may directly reach an event logging mode by activating a specific button. Further, the event is recorded by entering a code. Thus, the user may instantly enter the code representing the occurred event and will not need to respond to any questions from the apparatus. Further, the apparatus enables registration of a vast amount of different events, since they are each represented by a code. The number of different events allowed may then be determined by e.g. the number of bits used for storing the code in the memory, and not by the interface of the apparatus or the requests made by the apparatus.

Further, the recording of an event may be user-initiated. The user may bring the apparatus to enter the event logging mode and thereby initiate the possibility to record an event. This implies that the user may actually force the logging of an event exactly when it happens. He will not have to wait until the logging apparatus requests information about events to record an event which may have occurred several hours ago. Moreover, the diabetic may deviate from his normal routines and record the events and thereafter study the effect of the deviations. Thus, the need for creating a log of events does not restrict the diabetic in how to lead his life. Instead, it is possible to analyse the effects of deviations from normal routines and thus to improve the way of treating the diabetes.

The fact that the events are simple to record makes the diabetic more willing to use the apparatus. Therefore, the log will be more correct, since all relevant events will be logged. The log may thus be used to bring knowledge both to the diabetic and the physician on how to treat the disease and how the lifestyle of the diabetic affects the disease. This increases the possibility of giving good instructions on how to treat the diabetes, since the physician will have thorough data on which to base his instructions.

Since the apparatus is handheld, it may easily be carried along at all times by the diabetic. This also increases the possibility of creating a correct log.

Further, since the apparatus comprises a measuring means, the measured glucose level may be transferred to and recorded in the memory and thus be added to the log. Since the diabetic may measure the glucose level using the logging apparatus, he will not need to carry two separate devices with him at all times.

In the context of this application, the term “in direct response” should be construed as meaning that the apparatus will perform the response without a user needing to take any further action. The response may not be achieved instantly. However, even the response occurs a while after the activation, it will be a direct response if it is not dependent on any further action from the user.

The measuring means may be adapted to measure a glucose level in whole blood, in blood plasma or serum or in water phase of blood. The apparatus may use any method of measuring glucose levels in blood. The measured glucose level may depend on the measurement method, but as long as the measurement method is known, the information extractable from the log of glucose levels is not affected by the measurement method used.

The control circuitry may further be adapted to, in response to an activation of a specific button amongst the one or more buttons, directly enter an insulin dose logging mode, in which mode an administered insulin dose may be recorded in the apparatus. The treatment of diabetes is performed by administering insulin. Thus, recording the administered insulin dose is important in order to allow follow-up of the treatment. Therefore, there is provided a specific button for entering the insulin dose logging mode allowing simple and direct access to this mode. Since the user will need to record the administered insulin dose several times a day, the simple access to the insulin dose logging mode makes the apparatus more user-friendly.

The set of events may e.g. include dietary intake and exercise. The set may also include other events, such as the diabetic having a fever or being at a party (staying up late). The most common and important events affecting the blood glucose level may have predefined codes. Further, there may be provided predetermined codes, for which the user may define himself what event they are to represent. The user may define the event to be represented by a code prior to, or after, having recorded the occurrence of the event by means of the code in the apparatus. Thus, the codes which are to be used for representing events are predetermined, but the events which they represent need not be predefined.

The apparatus may further comprise a connector unit for enabling connection of the apparatus to a computer. Thus, the log recorded in the apparatus may be transferred to the computer for facilitating presentation of the log in a clear manner. This makes analysis of the log simpler and further provides a suitable tool for a physician to teach a diabetic how the lifestyle affects his well-being.

### BRIEF DESCRIPTION OF THE DRAWINGS

The application will now by way of example be described in more detail with reference to the accompanying drawings.

FIG. 1 is a view of a medical logging apparatus according to an embodiment of the invention.

FIG. 2 is a block diagram of the medical logging apparatus of FIG. 1.

FIG. 3 is a schematic view of the medical logging apparatus of FIG. 1 with a cuvette holder shown in a cuvette inserting position.

FIGS. 4-5 are views of a display of the medical logging apparatus during a measurement of a glucose level in a blood sample.

FIG. 6 is a view of the display of the medical logging apparatus in an event logging mode of the apparatus.

FIG. 7 is a view of the display of the medical logging apparatus in an insulin dose logging mode of the apparatus.

FIG. 8 is a flow chart of a method for recording an event in the medical logging apparatus.

FIG. 9 is a view of an application window of a software for processing and presenting a log on a computer.

### DETAILED DESCRIPTION OF A PREFERRED EMBODIMENT

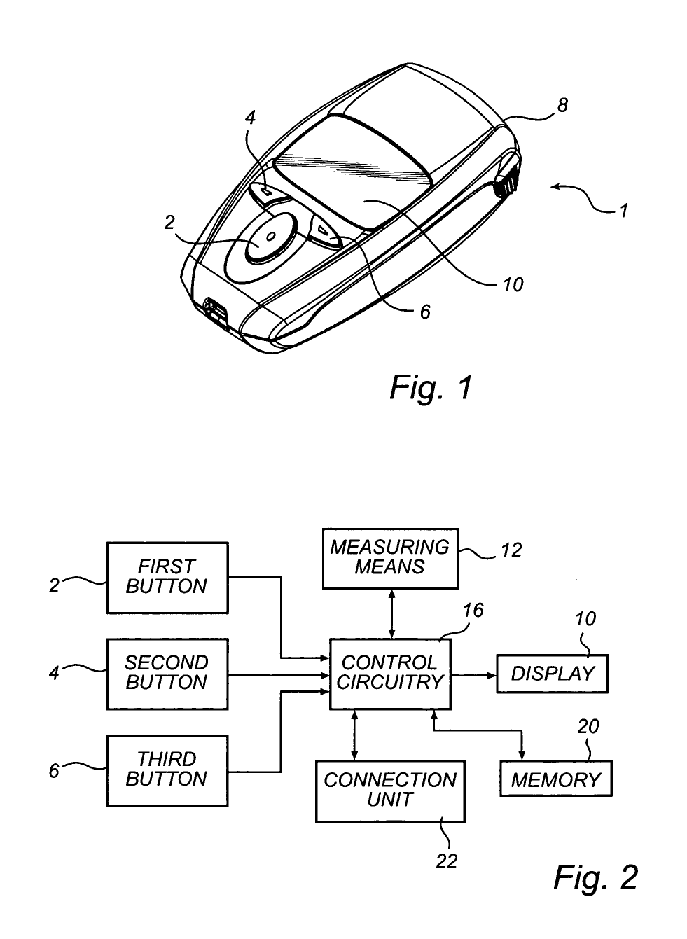

Referring now to FIG. 1, the medical logging apparatus 1 will be described. The medical logging apparatus 1 has a size which is suitable for holding it in the palm of a hand or carrying it in a pocket. Thus, it may easily be carried along at all times, without causing any inconvenience to a user. Further, it may easily be handled by the user holding the apparatus in one hand and operating the apparatus 1 with the other.

The medical logging apparatus 1 is used by the user for logging any events affecting the blood glucose level of the user. Thus, the medical logging apparatus 1 provides a possibility to measure the glucose level for logging the glucose level at a certain point of time, to record an administered insulin dose, and to record any other event that affects the blood glucose level. This implies that the user will be able to create a complete log of the variations of the glucose level and the events affecting the blood glucose level using merely a palm-sized apparatus.

The three different functions of the apparatus 1 are easily accessed. The apparatus comprises three buttons 2, 4, 6 for activating the three different functions. Thus, each function or operating mode may be initiated by activating the specific button 2, 4, 6. This implies that the operating mode is directly accessed, which makes the apparatus 1 very easy to use. This also provides the possibility to separately log a measured glucose level, an administered insulin dose and any other event affecting the blood glucose level. The user may thereby record the event when it actually occurred without needing to input any other information. Thus, upon activation from the user, the apparatus 1 enters a mode which is dedicated to performing the specific function requested by the user.

The function buttons 2, 4, 6 preferably each constitute a start-up button of the apparatus 1, starting up the apparatus 1 in the corresponding mode. However, the apparatus 1 may also include a separate start-up button. In this case, the function buttons 2, 4, 6 may merely initiate the apparatus 1 entering a specific mode.

The apparatus 1 comprises a first button 2 for bringing the apparatus 1 to enter the glucose level measurement mode. The apparatus 1 further comprises a second button 4 for bringing the apparatus 1 to enter an insulin dose logging mode. The apparatus 1 further comprises a third button 6 for bringing the apparatus 1 to enter an event logging mode.

The apparatus 1 has an outer shell 8, which protects inner, sensitive parts of the apparatus 1. The shell 8 has openings for the buttons 2, 4, 6 and for a display 10.

Referring now to FIG. 2, the functionality of the apparatus 1 will be described. The apparatus 1 comprises measuring means 12 for measuring the glucose level in a blood sample. The measuring means 12 comprises a cuvette holder 14 for receiving a cuvette with a blood sample.

The cuvette holder 14 is hingedly connected to the shell 8 of the apparatus 1 and is rotatably moveable between two positions. In a first cuvette receiving position, the cuvette holder 14 protrudes from the shell 8. In a second measuring position, the cuvette holder 14 is inserted into the shell 8 of the apparatus 1. The cuvette holder 14 then positions the cuvette with the blood sample correctly for the measurement.

The cuvette holds a reagent which interacts with the blood. The blood glucose and the reagent will form a colour which is dependent on the blood glucose level. The measuring means 12 is arranged to analyse the colour of the blood sample in order to determine the blood glucose level. Thus, the measuring means 12 further comprises a light source and a detector (not shown). The blood sample is irradiated by light from the light source and the detector detects light that has interacted with the blood sample. In this way, the colour of the blood sample may be determined and, thus, the blood glucose level in the blood sample is measured. The detector is connected to an analyzing unit (not shown), which analyses the detected light and calculates the blood glucose level. In U.S. Pat. No. 5,278,047, a measuring means which may be used in the apparatus 1 of the present invention is described in further detail.

The measuring means described above is adapted to measure a glucose level in whole blood. However, the apparatus 1 may instead comprise any other conventional measuring means adapted to measure a glucose level in blood plasma or serum or in water phase of blood. The measuring means may thus comprise a biosensor system, an electrochemical measuring system or a photometric measuring system for measuring the glucose level. Where the measuring means is adapted to determine a glucose level in blood plasma or serum or in water phase of blood, the actual “blood glucose level”, i.e. the glucose level in whole blood, is not determined, but instead a value reflecting the blood glucose level is determined.

It will be apparent to a skilled person that the measuring means may be modified in a number of different ways, while still allowing a value reflecting the blood glucose level to be determined.

Moreover, the apparatus 1 comprises control circuitry 16 which controls the functionality of the apparatus 1. The control circuitry 16 is operatively connected to and receives information of activations from the buttons 2, 4, 6. The control circuitry 16 processes the activations to bring the apparatus 1 into the corresponding mode. The control circuitry 16 then outputs to the display 10 a proper user interface for the selected mode. Further, the control circuitry 16 is, in a specific mode, arranged to interpret further button activations according to a specific scheme for the specific mode, as will be explained in further detail below.

The control circuitry 16 comprises a processor for handling the communication with other units of the apparatus 1. The control circuitry 16 further comprises a clock. The control circuitry 16 uses the clock to time and date stamp inputted data.

Thus, when an event has been inputted in the proper mode, the control circuitry 16 adds a time and date stamp to the event and transmits a signal to a memory 20 for storing the recording of the event.

Further, the apparatus 1 comprises a connection unit 22 for connecting the apparatus 1 to an external data processing unit, such as a personal computer, a mobile telephone or a personal digital assistant. The connection unit may comprise a USB-(Universal Serial Bus) port or any other means for establishing data transmission, such as a modem, an infrared transmitter/receiver, radio frequency transmitter/receiver using e.g. Bluetooth®, a network interface card for connection to a computer network, a cable, etc. This provides a possibility to transfer data from the apparatus 1 to the external data processing unit, which may be used for viewing the logged data.

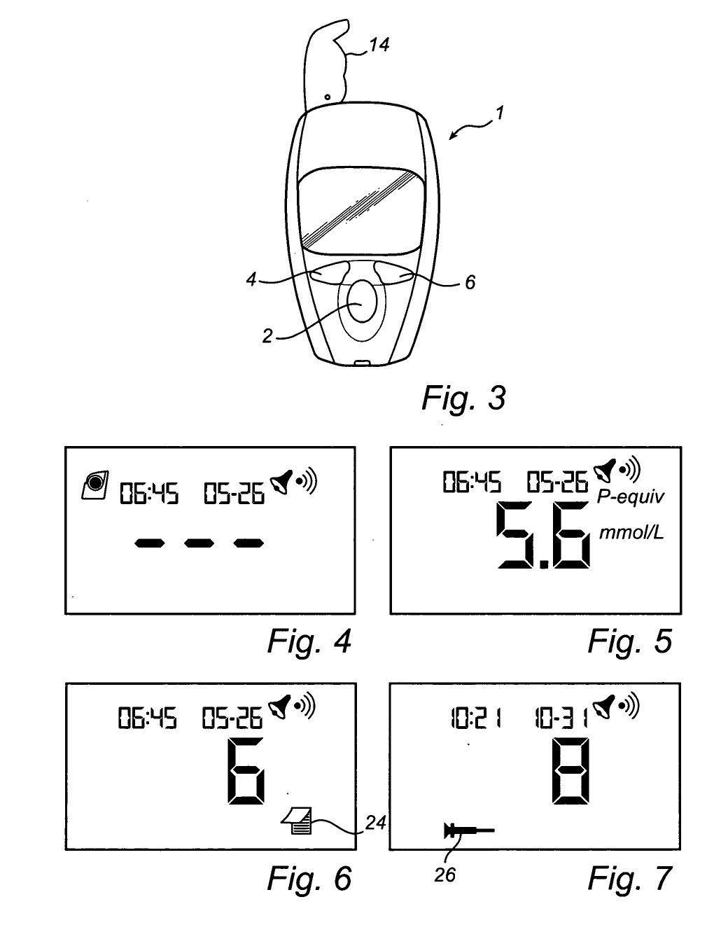

Referring now to FIGS. 3-5, the glucose level measurement mode will be described. Before starting the apparatus 1, a check should be made that the cuvette holder 14 is completely inserted into the measurement position. The glucose level measurement mode is initiated by activation of the first button 2. For a short while, there is displayed a start-up view showing information of the program version. Then, the latest measured glucose level is displayed with time and date for a short while. Thereafter, three flashing lines are displayed, as shown in FIG. 4. This signals to the user that the apparatus has entered the glucose level measurement mode and is ready for measurement.

Now, the cuvette holder 14 is to be extended by the user into the cuvette insertion position as shown in FIG. 3. The user then takes a blood sample using a lancer and a cuvette. The filled cuvette is applied in the cuvette holder 14. The cuvette holder 14 should be inserted into the measurement position as soon as possible (within 40 seconds) after being filled. The measurement is thus initiated. During measurement a sandglass and three fixed lines are shown to indicate that the measurement is being made. When the measurement is completed, the glucose level is shown in the display 10, as shown in FIG. 5. The value is displayed as long as the cuvette holder 14 is kept in the measurement position.

The control circuitry 16 receives the measured glucose level from the measuring means 12. The control circuitry 16 adds a time stamp to the glucose level and sends a data package containing the glucose level and the date and time of the measurement to the memory 20 where the measurement is stored. The cuvette may now be withdrawn from the cuvette holder 14 and the apparatus be shut off.

Referring now to FIG. 6, the recording of an event affecting the blood glucose level will be described. The apparatus 1 is started by pushing the third button 6. The apparatus 1 will then start up in the event logging mode. An event is recorded by means of a code. The apparatus 1 provides possibility to record any of a number of codes, which each represent a specific event. The codes may be numbers, preferably small, consecutive numbers. However, the codes may alternatively e.g. consist of one or a few letters. The events represented by a code may be predefined by the distributor of the apparatus 1. Suitably, the most common events are predefined. Further events to be represented by other codes may be user-defined. The user may define the event to be represented by a code prior to or after recording the code in the apparatus 1.

The codes representing predetermined events may, for example, be as follows:

| Code | Event |
|---|---|
| 1 | Breakfast |
| 2 | Lunch |
| 3 | Dinner |
| 4 | Snack |
| 5 | Evening |
| 6 | Exercise |
| 7 | Fever/illness |
| 8 | Party |

The user may add events by defining the events represented by codes of higher numbers. The way of defining the event a code represents will be described below.

When the user starts the apparatus 1 in the event logging mode, a note book symbol 24 is shown to indicate to the user that the apparatus has entered the event logging mode. A flashing code is shown on the display. The user may step forward to the correct code by repeatedly pushing the third button 6 or by keeping it pressed down. The code displayed may be decreased by pushing the second button 4 in similar manner. When the correct code is shown, the user confirms it by pushing the first button 2. The control circuitry 16 then sends the code with time stamp to the memory 20 for storing the occurred event. The apparatus 1 may now be shut off by keeping the first button 2 pressed down for a short while.

Referring to FIG. 7, the recording of an administered insulin dose will now be described. The apparatus 1 is started by pushing the second button 4. The latest administered insulin dose is now shown as long as the second button 4 is kept pressed down. When the button 4 is released, a flashing “0” will be shown. Further, a syringe symbol 26 will be displayed to indicate to the user that the apparatus 1 has entered the insulin dose logging mode. The number of units of administered insulin dose may now be recorded. The number displayed is increased or decreased in the same manner as described above for the codes in the event logging mode. The insulin dose may be recorded in integer units or in half units. When the correct number of units are displayed, the first button 2 is pressed down for confirmation. Now, the control circuitry 16 sends the information of the administered insulin dose with time stamp to the memory 20 for storage.

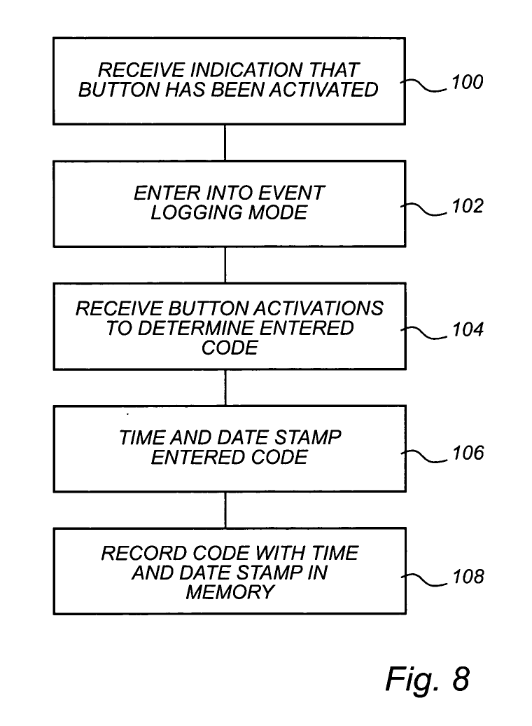

Referring now to FIG. 8, a method for recording an event in the medical logging apparatus will be described. First, the control circuitry receives an indication that a button has been activated, step 100. The control circuitry will identify which button has been activated. Where the third button has been activated, the apparatus is started and, in direct response to the activation, enters the event logging mode, step 102. The control circuitry then outputs to the display an indication to the user that the apparatus has entered the event logging mode.

The control circuitry receives in the event logging mode button activations made by the user, step 104. The button activations are interpreted by the control circuitry for determining an entered code representing an event. An activation of the second button is interpreted as decreasing the code number and an activation of the third button is interpreted as increasing the code number. The control circuitry continuously updates the display to indicate to the user which code that will be entered. When the control circuitry receives activation of the first button, the control circuitry determines the entered code to be the currently displayed code.

The control circuitry then time and date stamps the entered code, step 106. The entered code representing the occurred event is then recorded with the time and date stamp in the memory, step 108.

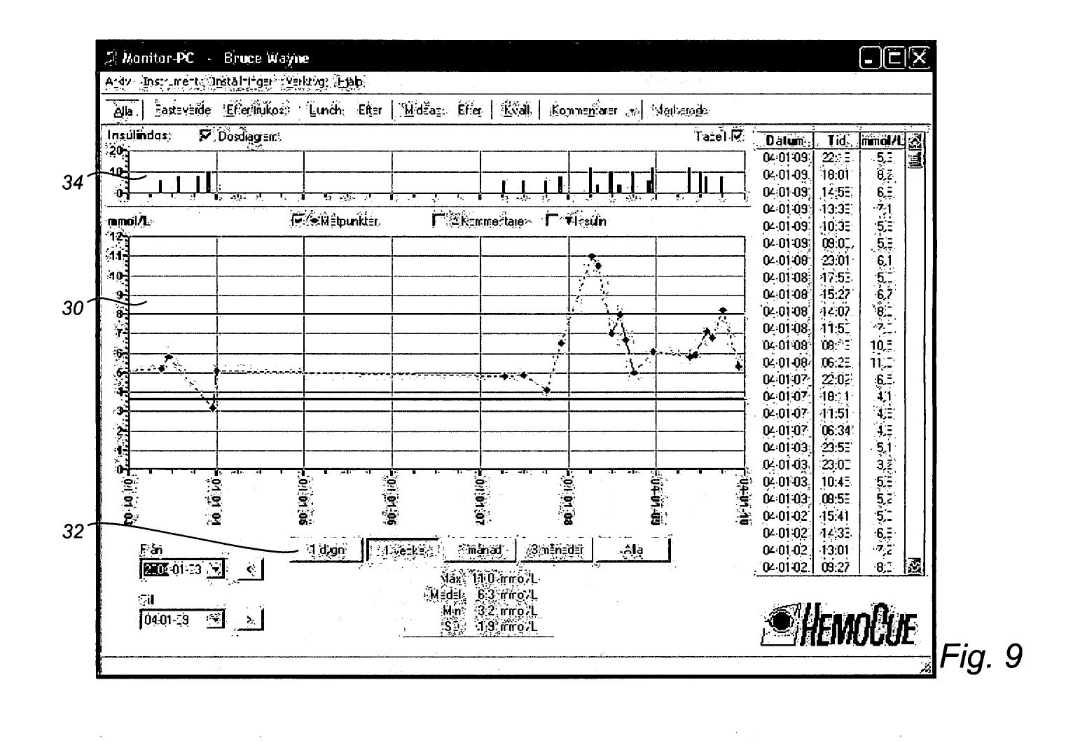

Referring to FIG. 9, the use of a computer application program for monitoring the log of the glucose level and the related events will be descried. The application program may be used by the diabetic for controlling and following up the variation of the glucose level. The application program could also be used by a physician for updating advice to the diabetic on how the diabetes should be treated. The use of the application program brings knowledge to the diabetic and the physician on how to treat the disease. The effects of the lifestyle of the diabetic on the illness are clearly illustrated, which allows the physician to pedagogically teach the diabetic how changes to the lifestyle will improve his well-being.

The recordings in the memory 20 of the apparatus 1 are transferred to the computer by connecting the apparatus 1 to the computer via the connection unit 22. This may, as explained above, be accomplished by means of a USB-cable or by other means, such as Bluetooth, infrared radiation, etc. The computer comprises an application program for handling the log.

The application program displays a chart 30 of the variation of the glucose level over time. The time period of the chart 30 may e.g. be chosen as 24 hours, one week, one month or three months or over the entire period of the log. The time period may be quickly changed by clicking the appropriate time period button 32. The chart 30 displays an upper and a lower limit, between which the glucose level should be. The upper and lower limits may be user-defined, i.e. defined by the diabetic or the physician. The times when the glucose level has been too high or too low may now easily be identified.

The administered insulin dose may be shown in a further chart 34, which is displayed with the same time axis as the glucose level chart 30. Thus, the glucose level may easily be related to the dosage scheme. Further, in the glucose level chart 30, the occurrence of an event may be indicated at the appropriate time. Thus, the glucose level may easily be related to a certain event. This implies that the effects of the different occurrences of events and the administered insulin dose may be easily viewed. The physician may therefore use the tool to analyse how the treatment of diabetes should be altered and to teach the patient what amendments to the lifestyle should be made.

There is a possibility to choose which glucose levels are to be displayed. Thus, the glucose level in relation to a specific event may be shown. In this way, the glucose level at, for instance, breakfast may be viewed. This provides analysis of the effect of a specific event, since the glucose level related to the specific event may be isolated and several such level values may be viewed to find a pattern of too high or too low glucose level.

It should be emphasized that the preferred embodiment described herein is in no way limiting and that many alternative embodiments are possible within the scope of protection defined by the appended claims. For example, the apparatus is described providing all user interaction by means of three buttons. The three buttons providing direct access to the different modes contributes to the simplicity of using the apparatus. However, the functions of entering a code or an insulin dose may be provided by further buttons.

The entering of a code in the event logging mode may be varied in a great number of ways. The code could e.g. be entered by using only one button. The code number is increased by activating the button. When the correct code is displayed, the button is kept pressed down for a while to indicate that this code is to be recorded. According to another alternative, a few codes may be displayed simultaneously. Then, the user may record the correct code by activating a button corresponding to, for example by being placed directly below the displayed code, one of the displayed codes. There may further be provided a button for requesting further codes.to be displayed. In this manner, the size of the display and the number of buttons will not limit the number of available events to be recorded.

1. A handheld medical logging apparatus for recording and logging data related to a person's blood glucose level, said apparatus comprising:
measuring means adapted to measure a glucose level in a blood sample,
one or more buttons,
control circuitry operatively coupled to the one or more buttons and adapted to, in response to an activation of a specific button amongst the one or more buttons, directly enter an event logging mode, in which mode an occurrence of one or more events from a set of events may be recorded in the apparatus, wherein each of said events may affect the blood glucose level and is represented by a predetermined code,
wherein the control circuitry, in the event logging mode, is adapted to interpret one or more activations of the one or more buttons as the code representing an event and create a signal indicative of the occurred event,
a memory operatively coupled to the control circuitry and adapted to receive the signal, and in response thereof record the occurrence of the event represented by the code.

2. The apparatus according to claim 1, wherein the measuring means is adapted to measure a glucose level in whole blood.

3. The apparatus according to claim 1, wherein the measuring means is adapted to measure a glucose level in plasma or serum.

4. The apparatus according to claim 1, wherein the measuring means is adapted to measure a glucose level in waterphase of blood.

5. The apparatus according to claim 1, wherein the control circuitry is adapted to, in response to an activation of a specific button amongst the one or more buttons, directly enter an insulin dose logging mode, in which mode an administered insulin dose may be recorded in the apparatus.

6. The apparatus according to claim 1, wherein the set of events include dietary intake and exercise.

7. The apparatus according to claim 1, further comprising a connector unit for enabling connection of the apparatus to a computer.

8. A method for recording and logging data related to a person's blood glucose level in a handheld medical logging apparatus comprising one or more buttons and a measuring means adapted to measure a glucose level in a blood sample, said method comprising:
receiving an indication in the apparatus that a specific button among the one or more buttons has been activated,
in direct response to the received indication, the apparatus entering an event logging mode, wherein an occurrence of one or more events from a set of events may be recorded in the apparatus, each of said events may affect the blood glucose level and is represented by a predetermined code,
in the event logging mode receiving in the apparatus one or more button activations specifying one of the codes representing an event, wherein the occurrence of said event is recorded in the apparatus.
